(19) United States
(12) Patent Application Publication
(10) Pub. No.: US 2009/0257973 A1
(43) Pub. Date: Oct. 15, 2009

(54) MALODOR REDUCING COMPOSITION, FRAGRANCE COMPOSITION AND PRODUCT COMPRISING THE SAME

(75) Inventors: Stuart FRASER, Little Neston (GB); Jonathan WARR, Paris (FR); Maud BASSEREAU, La Garenne Colombes (FR)

Correspondence Address: SUGHRUE-265550, 2100 PENNSYLVANIA AVE. NW, WASHINGTON, DC, 20037-3213, US

(73) Assignee: TAKASAGO INTERNATIONAL CORPORATION, Tokyo (JP)

(21) Appl. No.: 12/422,503

(22) Filed: Apr. 13, 2009

(30) Foreign Application Priority Data
Apr. 15, 2008 (EP) 08305104.5

(51) Int. Cl. A61L 9/01; A61K 8/49; C11D 3/50; A61Q 11/00; A61Q 13/00

(52) U.S. Cl. 424/76.1; 514/475; 512/11; 510/103

(57) ABSTRACT

The invention relates to a malodor reducing composition comprising:
at least one of (A) phenylglycidate represented by the following formula (1):

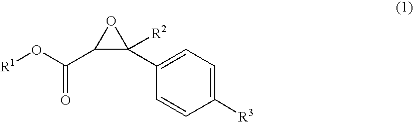

wherein R¹ is a C₁-C₄ branched or linear alkyl group, R² is hydrogen or methyl, and R³ is hydrogen, a C₁-C₄ branched or linear alkyl group or a methoxyl group;
and (B) 1,2-diketone represented by the following formula (2) or (3):

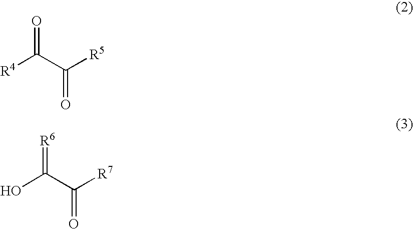

wherein R⁴, R⁵ and R⁷ are each independently, a C₁-C₅ linear or branched alkyl or alkenyl group, R⁶ is a (C₁-C₅) alkylidene, R⁴ and R⁵ may form a C₄-C₇ saturated or unsaturated alicyclic or hetrocyclic ring structure which may be mono-substituted or poly-substituted by (C₁-C₄) alkyl groups, and R⁶ and R⁷ may form a C₄-C₇ unsaturated, alicyclic or heterocyclic ring structure which may be mono-substituted or poly-substituted by (C₁-C₄) alkyl groups; and the weight ratio of the phenylglycidate (A) to the 1,2-diketone (B) being from 1:99 to 99:1. The invention also relates to the fragrance composition and consumer products containing this malodor reducing composition.

### CROSS-REFERENCE TO RELATED APPLICATIONS

This application claims priority from European Patent Application No. 08305104.5 filed on Apr. 15, 2008, the entire subject matter of which is incorporated herein by reference.

### BACKGROUND OF THE INVENTION

1. Technical Field

This invention is concerned with the use of certain fragrance ingredients to counteract malodors, especially human body odor, bathroom odors and odors from domestic waste. The invention also relates to fragrance composition and consumer products which incorporate such fragrance ingredients.

2. Background

The problem of malodors has been recognized for many years, and numerous methods have been developed to overcome these where they occur. Fragrances are commonly used as malodor counteractants either alone or in combination with other materials such as absorbents, oxidants and solubilisers.

Malodors are usually caused by particularly odorous substances such as sulfur compounds e.g. hydrogen sulfide, and low molecular weight thiols and thioeters, nitrogen containing compounds such as ammonia and amines including heterocyclic compounds such as pyrazines and indoles, and oxygen containing compounds such as short chain fatty acids and steroids. These malodorous compounds are often created by the action of microorganisms e.g. on food or drink during digestion, on sweat produced in the axilla or on the feet, on waste food awaiting disposal or animal waste such as cat litter. As natural products the malodors are themselves complex mixtures, not only of several compounds within each of the above chemical categories but also including members of all the categories to different degrees depending on the source of the odor and microorganisms involved. Consequently preventing, countering, reducing or destroying malodor is not straightforward.

Various methods have been developed to counter or reduce malodor beyond odor masking with strong fragrance. Activated carbon and zeolites have been used as absorbents for malodors but these are not suited for incorporation into many products. Antimicrobial agents such as zinc compounds and Triclosan (2′,4,4′-trichloro-2-hydroxydiphenyl ether) are used to kill microorganisms involved in producing malodors, but many are non-specific and can lead to imbalances in the natural microflora.

U.S. Pat. No. 4,304,679, U.S. Pat. No. 4,322,308, U.S. Pat. No. 4,278,658 and U.S. Pat. No. 4,134,838 all describe fragrances which exhibit a deodorant action when applied to human skin or when included in a laundry product. These are believed to work by any or all of a variety of mechanisms: reducing the vapor pressure malodorous compounds, inhibiting the enzymes which generate the malodors and combining with the malodor to modify the perceived odor note.

U.S. Pat. No. 4,719,105 describes the use of cyclomethanols and esters as low odor materials which can be used mask malodors without having unduly strong fragrances themselves.

US 2004/0147416 describes the use of cyclodextrins to trap malodorous molecules. However malodors tend to become stronger over time as the population of microorganisms grows and malodor production increases.

A different approach is to inhibit specific biological pathways. Compounds which act more specifically include saturated C₃-C₁₀ dioic acids which inhibit a sweat decomposing esterase as described in DE 4,343,265.

U.S. Pat. No. 6,183,731 describes the use of agents which are inhibitors of 5α-reductase, such as octadecendioic acid as a preventative agent for body malodor.

U.S. Pat. No. 5,395,555 teaches an aqueous cleaning composition for carpets, rugs, and textiles, particularly useful in reducing malodor of urine stains containing one of a group of selected metal sequestrants which is thought to deny heavy metals essential to the microorganisms' development.

However, as mentioned, earlier malodors are complex and such approaches may not counter all the components of the malodor, so it is more likely that they modify the nature of the odor which again may be a worthwhile benefit but is only a partial solution to the problem.

Aldehydes are sometimes cited as malodor counteractants. U.S. Pat. No. 4,906,454 describes piroctone acid and a perfume with a high concentration of aldehydes and/or ketones for use in deodorants in preventing body malodors. U.S. Pat. No. 6,177,070 describes a deodorant product containing at least two different aldehydes present in the form of addition compounds such as acetals and hemi-acetals and an unsaturated compound.

U.S. Pat. No. 5,676,163 describes two classes of aldehydes which act additively or synergistically to reduce the malodor of tobacco smoke. US 2005/187123 and US 2005/187124 describe compositions to be incorporated into solid and liquid carpet cleaner and deodorising products to remove malodors from carpets. US 2002/0010447 describes two categories of aldehydes for malodor reduction in fibrous absorbents for bodily fluids. The categories of aldehydes which are claimed to work synergistically are aliphatic aldehydes and aldehydes which have an sp2 hybridised carbon in the alpha position to the aldehyde carbon.

### SUMMARY OF THE INVENTION

Thus, while it is apparent from the related art that there are many methods which attempt to prevent malodor, none are completely satisfactory. The present invention describes a simple and inexpensive way to prevent the development of some of the components of malodor.

A summary of the invention is as follows.
(1) A malodor reducing composition comprising:

(A) phenylglycidate represented by the following formula (1):

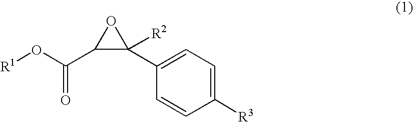

wherein R¹ is a C₁-C₄ branched or linear alkyl group, R² is a hydrogen atom or methyl group, and R³ is a hydrogen atom, a C₁-C₄ branched or linear alkyl group or a methoxyl group;

and (B) 1,2-diketone represented by the following formula (2) or (3):

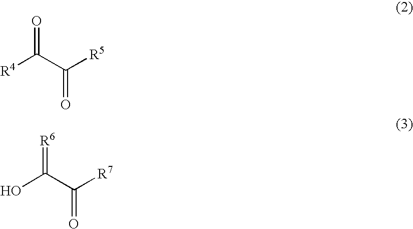

wherein R⁴, R⁵ and R⁷ are each independently a C₁-C₅ linear or branched alkyl or alkenyl group, R⁶ is a (C₁-C₅) alkylidene group, R⁴ and R⁵ may form a C₄-C₇ saturated or unsaturated alicyclic or hetrocyclic ring structure which may be mono-substituted or poly-substituted by (C₁-C₄) alkyl groups, and R⁶ and R⁷ may form a C₄-C₇ unsaturated, alicyclic or heterocyclic ring structure which may be mono-substituted or poly-substituted by (C₁-C₄) alkyl groups;

a weight ratio of the phenylglycidate (A) to the 1,2-diketone (B) being from 1:99 to 99:1.
(2) The malodor reducing composition according to (1), wherein the phenylglycidate is selected from the group consisting of ethyl 3-phenylglycidate (121-39-1), ethyl 3-methyl-3-phenylglycidate (77-83-8) and mixtures thereof.
(3) The malodor reducing composition according to (1) or (2), wherein the 1,2-diketone is selected from the group consisting of β-thujaplicine (499-44-5), 3-methylcyclopent-2-en-2-ol-1-one (765-70-8), 2,3-butanedione (431-03-8), 2-ethyl-3-hydroxypyran-4-one (4940-11-8) 2-methyl-3-hydroxypyran-4-one (118-71-8), ethyl cyclopentenolone (21835-01-8), 3,4-dimethyl-1,2-cyclopentadione (13494-06-9), 3,5-dimethyl-1,2-cyclopentadione (13494-07-0), 3-ethyl-2-hydroxy-4-methylcyclopent-2-en-1-one (42348-12-9), 2-hydroxycyclohex-2-en-1-one (10316-66-2), 1-methyl-2,3-cyclohexadione (3008-43-3), 1,2-cyclohexadione (765-87-7) and mixtures thereof.
(4) The malodor reducing composition according to (3), wherein the 1,2-diketone is selected from the group consisting of β-thujaplicine (499-44-5), 2,3-butanedione (431-03-8), 2-ethyl-3-hydroxypyran-4-one (4940-11-8), 2-methyl-3-hydroxypyran-4-one (118-71-8) and mixtures thereof.
(5) A fragrance composition comprising at least 0.05% by weight, preferably 0.1 to 15% by weight of the malodor reducing composition according to any one of (1) to (4) based on a total composition of the fragrance composition.
(6) The fragrance composition according to (5), which further contains more than 10% by weight of at least one of aldehydes and α,β-unsaturated aldehydes based on a total composition of the fragrance composition.
(7) A product comprising the malodor reducing composition according to any one of (1) to (4) in an amount of 0.00005% to 15% by weight, preferably 0.01% to 1.5% by weight based on a total composition of said product.
(8) The product according to (7), which is selected from the group consisting of air care products, personal cleansing and deodorizing products, household cleaning products including cistern blocks, rim blocks or liquid rim blocks, and laundry and fabric care products.
(9) A product comprising the fragrance composition according to (5) or (6) in an amount of 0.05% to 100% by weight based on a total composition of the product.
(10) The product according to (9), which is selected from the group consisting of personal cleansing products, household cleaning products including cistern blocks, rim blocks or liquid rim blocks, and laundry and fabric care products, and comprises 0.05% to 15% by weight of the fragrance composition based on a total composition of the product.
(11) The product according to (9), which is an air care product.
(12) The product according to (9), which comprises the fragrance composition in an amount of 0.05% to 1.5% by weight based on a total composition of the product.
(13) The product according to (9), which is selected from the group consisting of personal cleansing and deodorizing products, and household cleaning products including cistern and rim blocks and liquid rim blocks.
(14) A process for reducing a malodor, which comprises dispersing the malodor reducing composition according to any one of (1) to (4) contained in a product in an amount of 0.00005% to 15% by weight based on a total composition of the product into an air.
(15) A process for imparting a malodor reducing effect to a substrate, which comprises contacting the substrate directly with the malodor reducing composition according to any one of (1) to (4).
(16) A process for imparting a malodor reducing effect to a substrate, which comprises contacting the substrate with a product comprising the malodor reducing composition according to any one of (1) to (4) in an amount of 0.00005% to 15% by weight based on a total composition of the product.
(17) A process for reducing a malodor, which comprises dispersing the fragrance composition according to (5) or (6) contained in a product in an amount of 0.05% to 100% by weight based on a total composition of the product into an air.
(18) The process for reducing a malodor according to (17), wherein the fragrance composition is contained in the product in an amount of 0.05% to 1.5% by weight based on a total composition of the product.
(19) The process for reducing a malodor according to (17) or (18), wherein the product is selected from the group consisting of personal cleansing and deodorizing product, household cleaning products including cistern and rim blocks and liquid rim blocks.
(20) The process for reducing a malodor according to (17), wherein the product is an air care product.
(21) A process for imparting a malodor reducing effect to a substrate, which comprises contacting the substrate directly with the fragrance composition according to (5) or (6).
(22) A process for imparting a malodor reducing effect to a substrate, which comprises contacting the substrate with a product comprising the fragrance composition according to (5) or (6) in an amount of 0.05% to 100% by weight based on a total composition of the product.
(23) The process for imparting a malodor reducing effect to a substrate according to (22), wherein the fragrance composition is contained in the product in an amount of 0.05% to 1.5% by weight based on a total composition of the product.
(24) The process for imparting a malodor reducing effect to a substrate according to (22) or (23), wherein the product is selected from the group consisting of personal cleansing and deodorizing product, household cleaning products including cistern and rim blocks and liquid rim blocks.
(25) The process for imparting a malodor reducing effect to a substrate according to (22), wherein the product is an air care product.
(26) A process for producing a composition, which comprises mixing (A) phenylglycidate and (B) 1,2-diketone with a weight ratio of the phenylglycidate (A) to the 1,2-diketone (B) of from 1:99 to99:1,

wherein the phenylglycidate (A) is a compound represented by the following formula (1):

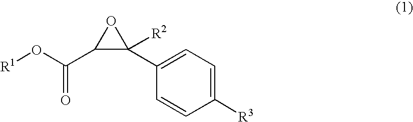

wherein R¹ is a C₁-C₄ branched or linear alkyl group, R² is a hydrogen atom or methyl group, and R³ is a hydrogen atom, a C₁-C₄ branched or linear alkyl group or a methoxyl group;

and the 1,2-diketone (B) is a compound represented by the following formula (2) or (3):

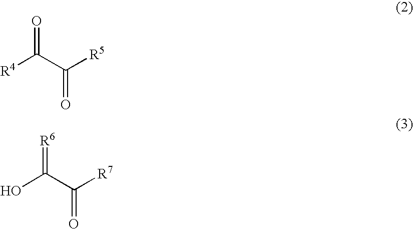

wherein R⁴, R⁵ and R⁷ are each independently a C₁-C₅ linear or branched alkyl or alkenyl group, R⁶ is a (C₁-C₅) alkylidene group, R⁴ and R⁵ may form a C₄-C₇ saturated or unsaturated alicyclic or hetrocyclic ring structure which may be mono-substituted or poly-substituted by (C₁-C₄) alkyl groups, and R⁶ and R⁷ may form a C₄-C₇ unsaturated, alicyclic or heterocyclic ring structure which may be mono-substituted or poly-substituted by (C₁-C₄) alkyl groups.

A further embodiment of the invention is a process for dispersing a malodor reducing fragrance which comprises at least 0.05% of the malodor reducing composition of the invention into ambient air in sufficient quantity as to reduce the perceived malodor.

Yet another embodiment of the invention is a process for imparting a malodor reducing effect to a substrate by contacting the substrate either directly with a malodor reducing fragrance which comprises at least 0.05% of the malodor reducing composition of the invention or by contracting the substrate with a consumer product containing a malodor reducing fragrance comprising at least 0.05% of the malodor reducing composition of the invention.

### DETAILED DESCRIPTION OF THE INVENTION

In this specification, all percentages quoted are weight percent unless otherwise stated. All documents cited in this specification are incorporated herein by reference. At the first mention compound chemical names are followed by the Chemical Abstracts reference number.

It has been found that phenylglycidates used in perfumery can counter many malodors such as human body odors, kitchen cooking toilet, urine, faeces, tobacco smoke and animal litter. However the phenylglycidates have relatively strong and specific odors so that they are not suited to be used at high level in a wide range of fragrances. Another group of chemical compounds which also counters malodors are 1,2-diketones. The 1,2-diketones have a different odor quality and, by varying the proportions of phenylglycidates and 1,2-diketones the combination thereof may be used at higher levels in a much wider range of fragrance types. Furthermore these combinations may act synergistically together and with other known malodor countering agents.

### Malodor Reducing Compositions

As used herein the term “malodor reducing compositions” mean combinations of phenylglycidates and 1,2-diketones, within the invention which reduce the concentration of malodors compounds in the air and/or reduces the perception of malodor to the human nose as measured by either chemical analysis or sensory methods.

### Malodor Reducing Fragrances

As used herein “malodor reducing fragrance” describes a fragrance composition which contains phenylglycidates and 1,2-diketones within the invention and which reduces the concentration of malodors compounds in the air and/or reduces the perception of malodor to the human nose as measured by either chemical analysis or sensory methods. The malodor reducing composition is contained in the fragrance composition preferably in an amount of at least 0.05% and more preferably 0.1% to 15% based on a total composition of the fragrance composition. A malodor reducing fragrance according to the present invention may also contain other fragrance compounds which are known as compounds countering malodors, such as aldehydes and α,β-unsaturated aldehydes in an amount of more than 10% by weight based on a total composition of the fragrance composition. Given the diversity of malodors it can be advantageous to design fragrances to counter specific odors.

### Malodor Reducing Ingredients

The main ingredients of the malodor reducing compositions and fragrances of the invention are defined in more detail herein below.

### Phenylglycidates

Phenylglycidates which have been found to counter malodors, have the general formula (1):

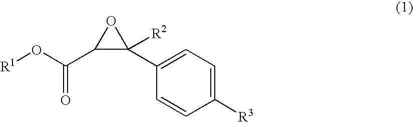

wherein:
R¹ is a C₁-C₄ branched or linear alkyl group,
R² is a hydrogen atom or methyl group, and
R³ is a hydrogen atom, a C₁-C₄ branched or linear alkyl group or a methoxyl group.

Example of suitable phenylglycidates include:
n-butyl 3-methyl-3-phenylglycidate (93963-69-0);
ethyl 3-(4-methoxyphenyl)glycidate (16546-01-3);
ethyl 3-(4-methyphenyl)glycidate (52788-71-3);
ethyl 3-phenylglycidate (121-39-1);
ethyl 3-methyl-3-phenylglycidate (77-83-8);
methyl 3-methyl-3-phenylglycidate (99334-18-6) and the like.

Two phenylglycidates which are particularly preferred for inclusion in fragrance compositions are: ethyl 3-phenylglycidate (121-39-1) and ethyl 3-methyl-3-phenylglycidate (77-83-8).

### 1,2-Diketones

Thus, 1,2-diketones which have been found to counteract malodors have the general formula (2) or formula (3) which is the enol form of formula (2):

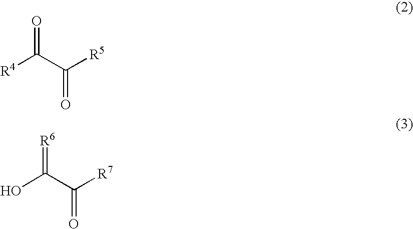

wherein:
R⁴, R⁵ and R⁷, which may be identical or different, represent a C₁-C₅ linear or branched alkyl or alkenyl group;
R⁶ is a (C₁-C₅) alkylidene group;
R⁴ and R⁵ may form a C₄-C₇ saturated or unsaturated alicyclic or hetrocyclic ring structure, which may be mono-substituted or poly-substituted by (C₁-C₄) alkyl groups;
R⁶ and R⁷ may form a C₄-C₇ unsaturated, alicyclic or heterocyclic ring structure which may be mono-substituted or poly-substituted by (C₁-C₄) alkyl groups.

Examples of such 1,2-diketones include:
2-hydroxy-4-isopropyl-2,4,6-cycloheptatrien-1-one also known as β-thujaplicine (499-44-5);
3-methylcyclopent-2-en-2-ol-1-one (765-70-8) called cyclotene which can also be found listed as the diketone tautomer 3-methyl cyclopentan-1,2-dione with the CAS number (80-71-7);
2,3-butanedione (431-03-8);
2,3-pentadione (600-14-6);
2-ethyl-3-hydroxypyran-4-one (4940-11-8);
2-methyl-3-hydroxypyran-4-one (118-71-8);
2,3-hexadione (3848-24-6);
3,4-hexadione (4437-51-8);
2,3-heptadione (96-04-8);
3-Ethyl-2-cyclopenten-2-ol-1-one (21835-01-8);
3,4-dimethyl-1,2-cyclopentadione (13494-06-9);
3,5-dimethyl-1,2-cyclopentadione (13494-07-0);
3-ethyl-2-hydroxy-4-methylcyclopent-2-en-1-one (42348-12-9);
2-hydroxycyclohex-2-en-1-one (10316-66-2);
1-methyl-2,3-cyclohexadione (3008-43-3) and
1,2-cyclohexadione (765-87-7).

Preferred examples of 1,2-diketone include β-thujaplicine (499-44-5), 3-methylcyclopent-2-en-2-ol-1-one (765-70-8), 2,3-butanedione (431-03-8), 2-ethyl-3-hydroxypyran-4-one 4940-11-8), 2-methyl-3-hydroxypyran-4-one (118-71-8), ethyl cyclopentenolone (21835-01-8), 3,4-dimethyl-1,2-cyclopentadione (13494-06-9), 3,5-dimethyl-1,2-cyclopentadione (13494-07-0), 3-ethyl-2-hydroxy-4-methylcyclopent-2-en-1-one (42348-12-9), 2-hydroxycyclohex-2-en-1-one (10316-66-2), 1-methyl-2,3-cyclohexadione (3008-43-3), 1,2-cyclohexadione (765-87-7) and mixtures thereof.

More preferred examples of 1,2-diketone include β-thujaplicine (499-44-5), 2,3-butanedione (431-03-8), 2-ethyl-3-hydroxypyran-4-one (4940-11-8), 2-methyl-3-one (118-71-8) and mixtures thereof.

The phenylglycidates and 1,2-diketones used according to the present invention are known commercially available products, or products which may be obtained by processes well known by the person skilled in the art. Bauer et al in Common Fragrance and Flavour Materials, 4^{th} Edition (ISBN 3-527-30364-2) gives brief descriptions and references for the preparation of the more common materials, which are incorporated herein by reference.

Examples of suitable processes for preparing 1,2-diketones are given hereinafter:

X-F Zhao and C Zhang, in Synthesis 2007 p 551-557, describes a method for the oxidation of 1,2-diols to 1,2-diketones;

Z Wan, C D Jones, D Mitchell, J Y Pu, and T Y Zhang in J. Org. Chem. 2006, vol 61, p 826-828 describes a method for oxidation of alkynes to 1,2-diketones;

J S Yadav, S K Biswas and R Srinivas in Synthesis 2006 p 4237-4241 describe a method for the oxidative cleavage of a 1,3-diol to give a 1,2-diketone.

Further references can be found in U.S. Pat. No. 4,107,210 assigned to Dow chemicals and references therein which describe commercial preparative routes to 1,2-diketones.

Glycidates are typically prepared either by epoxidation (Prileschaiev's reaction) or condensation (Darzens Glycidic Ester Condensation) reactions. These two synthetic processes for preparing phenylglycidates are described in standard organic chemistry reference books such as Organic Chemistry by I. L. Finar ISBN 0582442214.

The amount of diketones/phenylglycidates required for effective malodor reduction depends on the strength of the malodor, the type of product from which the fragrance is to be delivered, the product dose and the fragrance dose in the product. Given the wide range of products capable of delivering the malodor reducing composition and the range of fragrance dosage in consumer products, the dosage range of the malodor reducing composition can be from 0.00005% to 15%, more preferably at least 0.001%, and even more preferably 0.01% to 1.5% of the composition of the consumer product composition.

By composition of the consumer product composition is meant that part of the product which is expended in use; so excluding packaging and containers such as the devices for dispensing air care products, trigger sprays or canisters for pressurized aerosols and holders attached to toilet rims.

For household products for surface application, the amount of the malodor reducing composition is preferably between 0.00005% and 0.2% of the household product composition, and more preferably between 0.0005% and 0.1% of the usable composition.

For products used to treat ambient air, the proportion of the malodor reducing composition in the product composition is preferably between 0.0005% to 15% of the product composition, more preferably between 0.005% and 10% and especially preferably between 0.05% and 5%.

For personal care products for treating skin and hair, the proportion of the malodor reducing composition is preferably between 0.00005% and 0.2% of the overall personal care product composition, and more preferably between 0.0005% and 0.1% of personal care product composition.

For laundry detergent and laundrable fabric care products, the proportion of the malodor reducing composition is preferably between 0.0005% and 1.0% of the product, more preferably between 0.0001% and 0.5% and especially preferably between 0.0001% and 0.1%.

For personal cleansing products, household cleaning products, including cistern blocks, rim blocks or liquid rim blocks and laundry and fabric care products, the proportion of the fragrance composition of the invention is preferably at least 0.05% and more preferably between 0.05% and 15%.

For an air care product, the proportion of the fragrance composition of the invention is generally between 0.05% and 100%.

Phenylglycidates have relatively strong and specifically fruity odors hence their use in creating certain fragrance types may be limited. The diketones have a different odor quality and, by varying the proportions of phenylglycidates and 1,2-diketones the combination may be used at higher levels in a much wider range of fragrance types.

In the malodor reducing composition of the invention, the ratio of phenylglycidate to 1,2-diketone is from 1:99 to 99:1, preferably from 1:24 to 24:1, and more preferably from 1:9 to 9:1.

One embodiment of the invention is that the malodor reducing composition should comprise part of a malodor reducing fragrance. Even if the diketones/phenylglycidates are added to the product separately from other fragrance ingredients, they are considered part of the fragrance composition. Such a malodor reducing fragrance should contain at least 0.05% in total of the composition of phenylglycidates and 1,2-diketones of the invention, preferably at least 0.1% of the combination of phenylglycidates and 1,2-diketones and more preferably at least 0.2% of the combination of phenylglycidates and 1,2-diketones. While the diketones and phenylglycidates of the invention are especially effective at countering malodors, there are also other malodorants which may be present and for optimal counteraction it may be desirable to add ingredients which assist in malodor counteraction. Depending upon the source of the malodor and on the desired overall odor character of the fragrance, there will be an optimal balance between the phenylglycidate and 1,2-diketone combination and adding other perfume ingredients which may offer more general malodor counteraction properties. One category of fragrance ingredients which can be used very effectively in combination with the phenylglycidates and 1,2-diketones of the invention is aldehydes and α,β-unsaturated aldehydes.

### Fragrance Composition

In the context of this specification the word “fragrance” is understood to be synonymous with the word “perfume” and the terms “fragrance composition” or “perfume composition” and to refer to a mixture of olfactively active materials providing a pleasant smell. The term “fragrance ingredient” which is also synonymous with the terms “fragrance component”, “perfume ingredient” and “perfume component” is taken to mean any individual material which may be an ingredient within a fragrance composition even though that perfume ingredient may itself comprise many individual chemical compounds and possess a pleasant smell.

The fragrance composition to which the malodor reducing composition of the present invention may be added is a mixture, i.e. more than one chemical species, including ingredients which are known to be malodor counteractants. A wide variety of odiferous materials are known for perfumery use, including materials such as alkenes, alcohols, aldehydes, ketones, esters, ethers, nitrites, amines, oximes, acetals, ketals, thiols, thioketones, imines, etc. Without wishing to be limited, the ingredients of the fragrance composition may have molecular weights of less than 325 atomic mass units, preferably less than 300 atomic mass units and more preferably less than 275 atomic mass units to ensure sufficient volatility to be noticeable. Furthermore the fragrance composition may have molecular weights of greater than 50 atomic mass units, preferably greater than 60 atomic mass units as lower masses may be too volatile. Ingredients of the fragrance compositions will not contain strongly ionizing functional groups such as sulphonates, sulphates, or quaternary ammonium ions, nor halogen atoms.

Fragrance compositions containing the malodor reducing composition of the present invention may optionally contain natural extracts, such as essential oils. Natural extracts are produced by subjecting suitable natural materials such as plant components, for example leaves, flowers, seeds, roots or stems, to an extraction process. The extraction processes are well known to those skilled in the art and many are described in The Essential Oils by E Guenther published in 1949 by D van Nostrand. Essential oils can undergo additional processes to rectify and purify the oils, for example by removing the terpene components via a “head cut” and/or removing the wax components via a “tail cut”. Such natural extracts include but are not limited to those obtained from citrus species such as: lemons, oranges, mandarin, grapefruit, ugli fruit; from spices such as anise, cinnamon clove, or herbs such as basil, mint, lavender, lavandin, thyme, rosemary, or many varieties of plants such as geranium, various roses, citronella, cypress, eucalyptus, Peru balsam, camphor, sandalwood, ylang and cedarwood and mixtures thereof. A preferred group of national extracts for the present invention are Amyris oil, cedarwood oil, cocoa absolute, copaiba balsam, menthe oil pays, myrrh resin, patchouli oil, vanillin (absolute) and vetiver oil.

Fragrance compositions can be relatively simple in their composition with a minimum of three perfume or fragrance ingredients, including the phenylglycidates and 1,2-diketones, or they can comprise highly complex mixtures of natural and synthetic chemical components, chosen to provide any desired odor. It is preferable if the fragrance composition contains more than 5 fragrance ingredients, more preferable that they contain more than 10 fragrance ingredients. Fragrance ingredients are described more fully in S. Arctander, Perfume Flavors and Chemicals. Vol. I and II, Montclair, N.J. and in Allured's Flavor and Fragrance Materials-2007 ISBN 978-1-98263326-9 Allured Publishing Corporation.

### Solvents

Olfactively weak or neutral solvents may comprise part of the product. In the fragrance industry, it is quite common to dissolve solid fragrance materials in a suitable solvent or to dilute powerful materials, used at low levels, with a solvent to facilitate manufacture. Typical solvents include hydrophobic materials such as benzyl benzoate, isopropyl myristate, dialkyl adipates, citrate esters such as or aceryl tributyl citrate or diethyl phthalate or hydrophilic materials such as isopropanol, propylene glycol or dipropylene glycol or triethyl citrate. The common fragrance solvents which would preferably be used in consumer products containing malodor reducing compositions of this invention are described in S. Arctander, Perfume Flavors and Chemicals. Vols. I and II, Montclair, N.J. and in Allured's Flavor and Fragrance Materials-2007 ISBN 978-1-98263326-9 by Allured Publishing Corporation. Moreover solvents can also be beneficial in products, such as citrate esters being used to counter malodors in some personal care deodorant compositions, while other solvents can regulate fragrance evaporation and vapor pressure to achieve a desirable fragrance intensity and rate of fragrance delivery for air care products for example Isopar L® (Exxon Chemicals) and Dowanol DPM glycol ether® (The Dow Chemical Company). In this specification solvents are considered as part of the fragrance composition.

### Consumer Products

As used herein “consumer products” are defined as a functionally effective combination of ingredients which are consumed i.e. used up, in performing the task for which the product was intended such as cleansing, cleaning, deodorizing, sanitizing, softening, freshening, etc.

Examples of consumer products in which the malodor reducing composition or fragrance composition of the invention may be used are given hereinafter.

### (1) Personal Care Products

Shampoos, and hair conditioners, as exemplified by Head and Shoulders® and Pantene® (Procter & Gamble) or Elseve® (L'Oreal), shower gels, body wash as exemplified by Radox® (Sara Lee), liquid soap exemplified by Softsoap® (Colgate), soap bars as exemplified by Lux® or Dove® (Unilever), deodorant spray, roll-on and stick deodorants as exemplified by Rexona® or Sure® (Unilever), moist wipes including baby wipes, flushable moist toilet tissues, skin cleansing or refreshing wipes.

### (2) Laundry Detergents and Conditioners

Detergent powders, tablets, non soap detergent bars and hard soap bars, concentrated detergent powders, liquid detergents, concentrated liquid detergents, non-aqueous liquid detergents and unit dosing liquid detergent sachets. These may be exemplified by the following brands and variants Tide® and Aerial® (Procter & Gamble) Skip® and Omo® (Unilever) Persil® (Henkel). Fabric softeners or conditioners may be dilute or concentrated aqueous dispersions, or transparent liquids, powders or bars as exemplified by variants of Downy® (Procter & Gamble) Comfort® and Snuggle® (Unilever). Other post wash fabric treatments include tumble drier sheets such a Bounce® (Procter & Gamble) or ironing waters such as Comfort® (Unilever).

### (3) Household Cleaners

Household cleaners include liquid and spray hard surface cleaners which may be used directly on surfaces, liquids which can be diluted e.g. for floor cleaning or cleaning crockery and cooking utensils. Such products are exemplified by Flash® (Procter & Gamble), Cif® (Unilever) Fairy® Dishwash liquid (Procter & Gamble) or Pril® (Henkel). Unit dosed solid or liquid sachet or tabletted products such as for machine dishwashing as exemplified by Finish® (Reckitt Benckiser) or Fairy® Active Bursts (Procter & Gamble) and specialist cleaners for glass and mirrors e.g. under the brand Mr Muscle® (SC Johnson). These products are available in a variety of product forms such as sprays, mousses, liquids, liquids containing suspended solids, impregnated wipes sachets multicompartment sachets and unit dose devices. Bathroom sanitaryware cleaners include limescale cleaners, cistern blocks, rim blocks, liquid rim blocks and hybrid products exemplified by brands such as Harpic® (Reckitt Benckiser). Soft furnishing and fabric cleaners and refreshers include spray products such as Febreze® (Procter & Gamble) and carpet products such as Glade shake and Vac® (SC Johnson).

### (4) Air Care Products

Air care products include liquid electric powered air freshener devices, aerosol sprays, pump action sprays, fragranced candles, membrane permeation devices, Liquid wick devices, oil based gel fragrances, aqueous gels all of which may be exemplified by the following brands and variants Ambi Pur® (Sara Lee), Glade® and Oust® (SC Johnson) and Air Wick® (Reckitt Benkiser).

Such a diverse group of products contain a wide range of the proportion of fragrance within the formulation and are used at a wide range of product dosages which determine the levels at which the malodor reducing composition of the invention can be employed. Table 1 below gives typical fragrance dosages for a range of products in which the invention malodor reducing composition could be used.

TABLE 1
Typical Fragrance Dosages in Consumer Products

| | Typical Dosage Range |
|---|---|
| Product | (by weight) |
| Electric powered air freshener | 40-100% |
| Ambient temperature membrane devices | 40-100% |
| Ambient temperature fragranced oil gels | 40-85% |
| e.g. incorporating Sylvaclear PE400 | |
| polymer ® (Arizona Chemicals) | |
| Ambient temperature water based liquid wick | 5-20% |
| Aqueous freshener gels (e.g. carrageenan gels) | 2-10% |
| Liquid rim block, solid rim block, cistern block | 2-12% |
| Fragranced candles | 2-10% |
| Laundry detergent and conditioning products | 0.2-5% |
| Tumble drier sheets | 2-10% |
| Hard surface cleaning products | 0.1-0.5% |
| Toilet soaps, shampoos, hair conditioners, | 0.05-1.2% |
| shower gels, moist wipes | |
| Deodorant sprays, roll-ons or sticks | 0.05-1.0% |
| Air freshener trigger sprays and aerosols | 0.2-0.6% |

### Air Fresheners

One form of consumer products are air freshener devices to introduce fragrance to the ambient air in enclosed spaces such as rooms, vehicles, wardrobes, chests, drawers etc. These air freshener products may typically be in the form of sprays, candles, gels, membrane permeation devices, plug-in electrical devices, battery operated devices or liquid wick devices. In the present invention sprays may be aqueous or non-aqueous, pressurized with compressed gas or pomp action. Candles and gels may be opaque, translucent or transparent and may be moulded or packaged or contain additional ingredients to enhance their appearance. The plug-in and battery operated devices may include devices that vaporize the fragrance by heat, evaporation, or nebulisation.

Thus the process of dispersing the malodor reducing composition or fragrance containing the malodor reducing composition into ambient air may be by spraying, diffusion, evaporation or nebulisation.

Products for freshening or fragrancing general ambient air countering malodors or repelling insects include devices which have no power source and work by diffusion e.g. through a polymeric membrane, or by electrical heating a wick in contact with a liquid reservoir so that the rate of fragrance can be controlled. Such devices can be electrically powered, so called plug-in fresheners as described in U.S. Pat. No. 6,917,754 or U.S. Pat. No. 7,223,361 or WO 2007/046692 or battery powered so portable or for use in a motor vehicle. Optionally the device may use an atomizing spray to disperse the fragrance as in WO 2007/132140 or contain an electrically powered fan. Malodor reducing fragrance compositions of the present invention can be used advantageously in these freshener devices and compositions.

The present invention also provides a process to impart a malodor reducing fragrance to a substrate which may be skin, hair, soft furnishings, launderable textiles and garments, and hard surfaces including floors, walls, work surfaces toilets and bathroom sanitaryware.

The products from which the malodor reducing fragrances of the invention containing diketones and/or phenylglycidates may be delivered to skin and hair may be in the form of liquids, soft solids, gels, pastes, creams, powders, sprays or impregnated substrates such as pads or wipes. Liquids may include shampoos, conditions, shower gels, liquid soap, or lotions. The liquids may be clear opaque or pearlescent, of low viscosity or thickened to a gel-like viscosity. Soft solids include soap bars which includes mixtures of soap with synthetic detergents, such as alkyl isothionates or alkyl ethersulphates and deodorant sticks. Sprays include deodorant or antiperspirant sprays for skin or hair styling products and may be trigger action or pressurised canister type sprays. Impregnated substrates include wet wipes for personal hygiene, personal cleansing especially for babies and small children or refreshing. However the malodor reducing compositions of the invention are not intended for use in consumer products which are intended to be ingested such as foodstuffs or drinks, nor those which may be partially ingested as a result of the method of use such as in toothpaste or mouthwashes, nor products which may be accidentally ingested such as chewing gum.

The formulations and ingredients of personal care products in which malodor reducing compositions of the invention may be used are well known to those skilled in the art, reference may be made to the following works which are incorporated herein by reference:
“Formulating Detergents and Personal Care Products A guide to Product Development” by L Ho Tan Tai, ISBN 1-893997-10-3 published by the AOCS Press, and
“Surface Active Agents and Detergents” Volumes 1 and 2 by Schwarts, Perry and Birch, and
“Harry's Cosmeticology” published by CHS Press 8^{th} Edn 2000 ISBN 0-8206-0372-4, and
McCuthcheon's Detergents and Emulsifiers published by Allured and
Cosmetic Science and Technology Series Volume 20, Edited by K. Laden, (Marcel Dekker) ISBN 0-8247-1746-5 “Antiperspirants and Deodorants” and
Cosmetic Science and Technology Series Volume 17, Ed by Dale H Johnson, ISBN 0-8247-9365-X “Hair and Hair Care” published by Marcel Dekker, as well as to the following patents or patent applications; all of these patents and the references therein are incorporated herein by reference.

### Shampoos and Hair Conditioners:

U.S. Pat. No. 6,162,423; U.S. Pat. No. 5,968,286; U.S. Pat. No. 5,935,561; U.S. Pat. No. 5,932,203; U.S. Pat. No. 5,837,661; U.S. Pat. No. 5,776,443; U.S. Pat. No. 5,756,436; U.S. Pat. No. 5,661,118; U.S. Pat. No. 5,618,523; EP 0,018,717; EP 1,009,365; EP 0,200,305.

### Wipes:

WO 2003/051327; WO 00/04230; EP 1,361,855; US 2005/0008680;

### Skin Cleansing and Care Liquids:

U.S. Pat. No. 5,833,999; EP 1,066,827; EP 1,510,201; EP 0,573,229; US 2005/0085405;

### Laundry Products

Malodor reducing compositions of the invention might advantageously be employed in products for domestic cleaning of washable textiles such as clothes, towels, and bed linen. Suitable laundry products include: solid bar, powder, tablet, and liquid detergents, fabric softeners and non-softening fabric conditioning products. Also included are post wash laundry treatment products such as ironing waters, garment refresher sprays and articles impregnated with cleaning or conditioning liquids such as tumble drier sheets. The formulation of laundry products if familiar to those skilled in the art and reference may be made to “Formulation Detergents and Personal Care Products A guide to Product Development” by L Ho Tan Tai, and to “Surface Active Agents and Detergents” Volumes 1 and 2 by Schwartz, Perry and Birch both referenced earlier and also to Volume 67 of the Surfactant Science Science Series Liquid Detergents ISBN 0-8247-9391-9 (Marcel Dekker Inc), as well as to the following patents or patent applications:

### Liquid Laundry Detergents:

U.S. Pat. No. 5,929,022; U.S. Pat. No. 5,916,862; U.S. Pat. No. 5,731,278; U.S. Pat. No. 5,470,507; U.S. Pat. No. 5,466,802; U.S. Pat. No. 5,460,752 and U.S. Pat. No. 5,458,810.

### Detergent Powders and Tablets:

WO 99/65458; WO 99/41353 and EP 1,123,381.

### Fabric Softeners and Conditioners:

U.S. Pat. No. 6,627,598; U.S. Pat. No. 6,335,315; U.S. Pat. No. 5,674,832; U.S. Pat. No. 5,759,990; U.S. Pat. No. 5,877,145; U.S. Pat. No. 5,574,179.

The products from which the malodor reducing fragrances containing diketones and/or phenylglycidates of the invention may be delivered to domestic soft furnishings such as curtains, carpets, or upholstery or hard surfaces such as wooden or tiled floors, walls, windows, kitchen worktops and bathroom sanitaryware, may be in the form of liquids, soft solids, gels, pastes, foams, creams, powders, sprays or impregnated substrates such as pads or wipes. Products may be applied as directly to surfaces, or diluted with water prior to use, or they may be incorporated in a device such as a cistern block or liquid rim block which is activated by flushing the lavatory.

### Lavatory Solid Rim Blocks and Cistern Blocks

One embodiment of the present invention relates to malodor reduction on hard surfaces and sanitaryware. Lavatory blocks including those which are intended, in use, to be located under the rim of a lavatory bowl or urinal such that, during a flushing cycle, water from the cistern flows over the block thereby dissolving a portion of the block are a particular example of a product in which the malodor reducing compounds of the invention can be used. Such blocks are generally known in the art as ‘rim blocks’ and will be referred to as such or simply as ‘blocks’ herein. The invention also relates to so-called cistern blocks which are placed in the cistern and dissolve slowly in the water contained therein. It will be appreciated that the solubility characteristics of these two products are quite different, since one is constantly under water while the other has intermittent short term contact with water. However they both contain a surfactant, filler, and fragrance which may contain ingredients of the present invention and optionally bleaching agents, germicides and anti-limescale agents. Typical formulations are taught in WO 97/47721; EP 462,643; GB 2,178,442 and U.S. Pat. No. 4,874,536 all of which are incorporated herein by reference.

### Liquid Rim Blocks

Another embodiment of the malodor reducing compounds of this invention relates to lavatory cleaners known as liquid rim blocks. Liquid rim blocks are devices that dispense liquid compositions directly into a lavatory bowl from under the rim of said bowl. Such rim blocks are usually attached by various means, such as hooks and the like, to the rim of the lavatory bowl. Every time a toilet equipped with a rim block is flushed, an amount of composition is dispensed into the lavatory bowl. Examples of liquid rim blocks are given in WO 02/40792 which teaches a liquid rim-block device having a suspension means and at least two compartments for active substances. WO 02/04591 teaches a liquid rim block, a lavatory cleaning system comprising a dispenser for dispensing a liquid composition from under the rim of a lavatory bowl, wherein the composition has a viscosity of greater than 2500 mPa·s. Other similar systems are described in EP 775,741 and WO 01/94520 all of which are incorporated herein by reference.

Rim blocks may also comprise combinations of solid and liquid within a single device. Such devices are commercially available for example under the trade name Harpic® from Rickitt Beckiser.

### Liquid Hard Surface Cleaner Formulation

Another specific application of malodor reducing fragrances of the invention relates to hard surface cleaner formulations also known as all purpose cleaners or general purpose cleaners. They are a broad category of products including isotropic liquid, thickened liquid with or without abrasive or as a mousse. They can be used directly from the bottle or after dilution in water. Various delivery methods have been devised for the convenience of the user, some are sprayed onto surfaces from trigger spray bottles, and alternatively they can be poured directly onto surfaces and removed for instance when a lavatory is flushed. They may contain additional ingredients such as acids for limescale removal, biocides for hygiene, bleaches. Consequently there are a broad range of formulations within this category. Table 2 below summarises the main formulation ingredients and levels (this is taken from Surfactant Science Series Vol 67 Liquid Detergents chapter on Speciality liquid Household Surface Cleaners p 479 table 4.) In all cases, except when oxidizing bleaches (e.g. sodium hydrochlorite or hydrogen peroxide) are incorporated into the formulation, malodor reducing fragrances containing phenylglycidates and 1,2-diketones of this invention may be used as the fragrance within the product.

TABLE 2
Typical Formulation Ranges for Ingredients
of Household Cleaners

| | | Amount |
|---|---|---|
| Ingredient | Example | wt % |
| Anionic | Alkylbenzene sulphonate, as supplied by Shell | 0-35 |
| surfactant | as Dobs 055, alkane sulphonate eg Hostaspur | |
| | SAS60 | |
| Nonionic | Ethoxylated alcohol, e.g.: Neodol 9-11 6EO; | 1-35 |
| surfactant | mixed ethoxy/propoxy alcohol such as the | |
| | pluronic series from BASF, amine oxides, | |
| | alkanolamides and betaines | |
| Hydrotropes | Sodium cumene sulphanate or xylene sulphanate. | 0-10 |
| Builder/ | Citrates, EDTA salts; phosphonate salts; lactic | 0-10 |
| sequestrant | acid and polyacrylates. | |
| Solvent | Lower alcohols; glycol ethers; benzyl alcohol or | 0.5-50 |
| | hydrocarbons, e.g. limonene. | |
| Disinfectant | Hydrochlorite bleach; pine oil; lower alcohols; | 0-15 |
| | quaternary ammonium salts. | |
| Fragrance; | | 0.1-3 |
| cooling | | |
| agent; | | |
| thickening | | |
| polymer; | | |
| sequestrant; | | |
| preservatives | | |
| Water | | To 100 |

### Carpet Cleaners

Carpet cleaners come in several forms: powders, liquids, foams and spray spot treatments. Many of these products have to be used in conjunction with a specific cleaning machine especially if large areas are to be treated. However a desirable attribute of cleaning a carpet is to reduce malodors and leave a pleasant smell, such products generally contain a fragrance. Malodor reducing fragrances of the present invention are both suitable and desirable for application in carpet cleaning products.

### Air Freshener, Textile and Soft Furnishing Deodorizing Sprays

Products for reducing or masking malodors in the air or on textiles and soft furnishings are known, such as Glade® (SC Johnson) and Febreze® (Procter and Gamble). These products are designed to be sprayed into the air or onto surfaces. They can be dispensed either from aerosols with a volatile propellant gas or using a trigger spray which does not require propellant gas. US 2005/0124512 teaches a cyclodextrin containing product in which non fabric discolouring aldehydes are incorporated to react with amines in the air. Nowhere does the patent teach that phenylglycidates or 1,2-diketones either separately or combined might inhibit malodor formation. US 2003/0044309 teaches as emulsion composition containing a selection of fragrance aldehydes which counter malodor. Fragrances incorporating phenylglycidate and 1,2-diketones as malodor reducing compounds can be used advantageously in these products.

The following examples are provided to further illustrate the compositions and processes in accordance with the invention. The examples are illustrative only and are not intended to limit the scope of the invention in any way.

### REFERENCE EXAMPLE 1

The malodor reducing capability of individual ingredients, namely 1,2-diketones and phenylglycidates of the invention is tested according to the following procedure.

### Procedure

Add into a 20 cm³ headspace vial:
   100 μg of a 1% (wt/wt) solution of 1-hexylamine in dipropylene glycol;
   7.0 g of demineralised water;
   20 μl of 1,2-diketone or phenylglycidate;
   0.2 g of Emulgin L® (Cognis)
Mix for 10 minutes at room temperature;
Equilibrate for 50 minutes at 35° C.;
Measure the headspace concentration of the hexylamine by solid phase microextraction (SPME) on a polydimethylsilicone divinylbenzene fibre (ex Supelco);
Sample the vapour for 5 minutes at 35° C. then desorb onto a 30 m HP-5MS GC column at 265° C. for 1 minute.

The headspace concentration is calculated by comparison with a standard having no malodor reducing fragrance and is reported as the reduction in area percent of the GCMS signal for hexylamine. The results are given in Table 3.

TABLE 3
Malodor reduction by compounds of the invention

| Ingredient (CAS Number) | Malodor reduction % |
|---|---|
| β-Thujaplicine (499-44-5) | 97 |
| 2,3-butanedione (431-03-8) | 82 |
| 3-methylcyclopent-2-en-2-ol-1-one (765-70-8) | 66 |
| 2-methyl-3-hydroxypyran-4-one (118-71-8) | 71 |
| Ethyl 3-methyl-3-phenylglycidate (77-83-8) | 41 |
| Ethyl 3-phenylglycidate (121-39-1) | 63 |

### REFERENCE EXAMPLE 2

This reference example shows the effect of increasing the concentration of a typical diketone β-thujaplicine within fragrance A of table 4 on the malodor compared with the fragrance itself. The procedure is similar to that described in reference example 1 above except that a 50 μl aliquot of fragrance is used instead of the 20 μl aliquot of the 1,2-diketone or phenylglycidate in reference example 1. The test fragrance consists partly of fragrance A and partly of a 1,2-diketone, phenylglycidate or a mixture thereof, with part of the diethyl phthalate within fragrance A being replaced by the malodor reducing compounds of the invention. Malodor reduction is measured as the concentration of hexylamine in the vapour phase compared with a control sample which contains fragrance A without any malodor reducing ingredients of the invention.

TABLE 4
Formulation of Fragrance A

| Ingredient | CAS No | % |
|---|---|---|
| Allyl cyclohexyl propionate | 2705-87-5 | 0.2 |
| Allyl heptoate | 142-19-8 | 0.3 |
| Armoise oil | | 0.4 |
| Benzyl acetate | 140-11-4 | 4.1 |
| Benzyl Salicylate | 118-58-1 | 12.0 |
| Dihydromyrcenol | 18479-58-8 | 10.0 |
| Dimethyl anthranilate | 85-91-6 | 0.1 |
| Eugenol | 97-53-0 | 0.3 |
| Florosa | 63500-71-0 | 3.0 |
| Geraniol | 106-24-1 | 4.2 |
| Guaicawood oil | | 0.6 |
| Hedione | 24851-98-7 | 5.0 |
| Cis 3-hexenyl acetate | 35926-04-6 | 0.5 |
| Isobornyl acetate | 78-70-6 | 10.0 |
| Linalool | 78-70-6 | 10.0 |
| Mandarin oil | | 0.6 |
| Methyl ionone gamma A | 127-51-5 | 0.7 |
| Ethylene brassylate | 105-95-3 | 6.0 |
| Patchouli oil de-ironised | | 0.2 |
| Phenyl acetaldehyde Dimethyl acetal | 101-48-4 | 0.2 |
| Phenyl ethyl alcohol | 60-12-8 | 5.2 |
| Rose oxide | 16409-43-1 | 0.2 |
| Styrallyl acetate | 93-92-5 | 0.9 |
| Undecalactone gamma | 104-67-6 | 0.3 |
| Verdox | 88-41-5 | 0.3 |
| Diethyl phthalate | 84-66-2 | 25.0 |

Fragrance A is a citrus floral rose fragrance suitable for use in laundry or personal care products.

TABLE 5
Malodor reduction due to dosage of β-thujaplicine

| β-thujaplicine in Fragrance A* | Malodor Reduction (%) |
|---|---|
| 1% | 47 |
| 2.5% | 76 |
| 5% | 93 |

*β-thujaplicine replaces the equivalent weight of diethyl phthalate in fragrance A. So 1% β-thujaplicine mean only 24.0% of dimethyl phthalate is used to make up that test sample

### REFERENCE EXAMPLE 3

Reference example 3 repeats reference example 2 but with ethyl 3-phenylglicidate instead of β-thujaplicine. The results in table 6 show a definite trend in response to increasing the phenylglycidate concentration.

TABLE 6
Malodor reduction due to dosage of Ethyl 3-phenylglicidate

| Ethyl 3-phenylglicidate in Fragrance A** | Malodor Reduction (%) |
|---|---|
| 1% | 31 |
| 5% | 39 |
| 10% | 54 |

**Ethyl 3-phenylglicidate replaces the equivalent weight of Diethyl Phthalate in fragrance A as described in reference example 2.

### EXAMPLE 1

Example 1 repeats reference example 2 but with a range of mixtures of ethyl 3-phenylglycidate and 2-methyl-3-hydroxypyran-4-one (118-71-8). The ratio of ethyl 3-phenylglycidate to 2-methyl-3-hydroxypyran-4-one was used between 9:1 to 1:9 by weight percent, with the total weight of the combination always representing 3.0% of fragrance A. The results show that the combination gives greater malodor reduction than ethyl 3-phenylglycidate at 5% (cf table 6 with table 7) and it equal to the ethyl 3-phenylglycidate at 10% by weight of fragrance A. Furthermore the change in malodor reduction is not particularly sensitive to the ratio of ingredients

TABLE 7
Malodor reduction due to Combinations of Ethyl
Phenylglycidate and 2-Methyl-3-hydroxypyran-4-one

| Ratio of Ethyl 3-phenylglicidate:2-methyl-3- | |
|---|---|
| hydroxypyran-4-one*** | Malodor Reduction (%) |
| 1:9 | 68 |
| 25:75 | 69 |
| 50:50 | 49 |
| 75:25 | 54 |
| 9:1 | 54 |

***Each mixture of Ethyl 3-phenylglicidate and 2-methyl-3-hydroxypyran-4-one replaces 3.0% of Diethyl phthalate in fragrance A.

### EXAMPLE 2

A fragrance composition in the form of a gel for a membrane air care device or an electrically powered air care device is prepared by mixing 40% of the fragrance composition of table 8 with 55.95% of Dipropylene glycol monomethyl ether, 4% of fumed hydrophilic silica and 0.05% of polyoxyethylene sorbitan monolaurate.

TABLE 8
A Perfume Composition for Gel or
Electrically Powered Air Care Device

| | | | % by |
|---|---|---|---|
| Ingredient | Chemical name | CAS No | weight |
| HEDIONE | Methyl dihydrojasmonate | 24851-98-7 | 35 |
| | Ethyl 3-methyl-3-phenyl- | 77-83-8 | 1.125 |
| | glycidate | | |
| β-THUJAPLICINE | | 499-44-5 | 1.875 |
| (1% in DPG) | | | |
| ORBITONE | Octahydro-2,3,8,8-tetra- | 54464-57-2 | 32.5 |
| | methyl-2-acetonaphtone | | |
| DIHYDRO | 2,6-dimethyl-7-octen-2-ol | 18479-58-8 | 10.25 |
| MYRCENOL | | | |
| MUSK-T | 1,4-Dioxacyclohepta- | 105-95-3 | 15 |
| | decane-5,17-dione | | |
| LEVOSANDOL | 2-ethyl-4-(2,2,3-trimethyl- | 28219-61-6 | 2 |
| | 3-cyclopenten-1-yl)-2- | | |
| | buten-1-ol | | |
| CITRAL | 3,7-dimethyl-2,6-octadienal | 5392-40-5 | 1.25 |
| | (mixture of cis and trans) | | |
| ALLYL | Allyl | 2705-87-5 | 0.25 |
| CYCLOHEXYL | cyclohexanepropionate | | |
| PROP | | | |
| AMBROXAN | Dodecahydro-3a,6,6,9a- | 3738-00-9 | 0.25 |
| | tetramethylnaphtho-(2,1-b)- | | |
| | furane | | |
| AMBRETONE | 5-cyclohexadecan-1-one | 37609-25-9 | 0.25 |
| HEXENYL | Cis hex-3-en-1-yl acetate | 3681-71-8 | 0.125 |
| ACETATE, | | | |
| CIS-3 | | | |
| | Ethyl 2-methylbutyrate | 7452-79-1 | 0.125 |

The malodor reducing composition comprises 3.0% of the fragrance and 1.2% of the final product composition with a ratio of phenylglycidate to 1,2-diketone of 1:1.7. Such a fragrance can be used in devices for the diffusion of ambiance-generating fragrances, which are available from commercial sources, such as those known under the trade names “Glade® Plug-In”, “Glade® Wisp”, “Recitt-Benckiser Air-Wick® Mobil”, “Air-Wick®”, “Ambi-Pur Car®” and “Sara Lee Inspira®”.

### EXAMPLE 3

### Perfumed Candle

A perfuming candle was prepared by melting the wax and the other ingredients in table 9 below a water bath at 80° C. and then adding the perfuming oil according to table 10 and mixing until a uniform mixture was obtained, which was then flowed into a mould with a wick.

TABLE 9
Candle Composition

| NAME | % |
|---|---|
| PARAFFIN WAX 52.54 (AIGLON SA) | 22 |
| STEARIC ACID PRISTERENE 9559 (UNIQUIMA) | 20 |
| BEEWAX, CEREWAX A.75 (BAERLOCHER FRANCE) | 8 |
| WHITE VASELINE (AIGLON SA) | 16 |
| LIQUID PARAFFIN (AIGLON SA) | 24 |
| PERFUME COMPOSITION OF TABLE 10 | 10 |

The candle thus formed was then left to cool down for 24 hours. This candle was then burnt so as to diffuse the fragrance composition in a stable way during at least 24 to 36 hours.

TABLE 10
Perfuming Oil for Candle

| Ingredient | Chemical name | CAS No | % |
|---|---|---|---|
| HEDIONE | Methyl dihydrojasmonate | 24851-98-7 | 29.0 |
| LINALOOL | 3,7-dimethyl-1,6-octadien-3-ol | 78-70-6 | 18.2 |
| MUSK-T | 1,4-Dioxacycloheptadecane-5, | 105-95-3 | 12.3 |
| | 7-dione | | |
| VERTENEX | 4-(1,1-dimethylethyl)- | 32210-23-4 | 12.3 |
| | cyclohexanol acetate | | |
| DIHYDRO | 2,6-dimethyl-7-octan-2-ol | 18479-58-8 | 7.4 |
| MYRCENOL | Ethyl-3-methyl-3-phenyl- | 121-39-1 | 2.0 |
| | glycidate | | |
| Veltol plus | 2-ethyl-3-hydroxypyran-4-one | 4940-11-8 | 0.3 |
| CITRAL | 3,7-dimethyl-2,6-octadienal | 5392-40-5 | 3.1 |
| | (mixture of cis and trans) | | |
| ALLYL | Allyl cyclohexyl-3-propionate | 2705-87-5 | 2.5 |
| CYCLOHEXYL | | | |
| PROP | | | |
| CITRONELLYL | (3R)-3,7-dimethyloct- | 51566-62-2 | 2.4 |
| NITRILE, L | 6-enenitrile | | |
| LEVOSANDOL | 2-ethyl-4-(2,2,3-trimethyl-3- | 28219-61-6 | 1.2 |
| | cyclopenten-1-yl)-2-buten-1-ol | | |
| ORBITONE | Octahydro-2,3,8,8-tetramethyl- | 54464-57-2 | 2.5 |
| | 2-acetonephthone | | |
| AMBROXAN | Dodecahydro-3a,6,6,9a-tetra- | 3738-00-9 | 0.2 |
| | methyl-naphtho (2,1-b) furane | | |
| DIMETHYL | | 10094-34-5 | 0.2 |
| BENZYL | | | |
| CARBINYL | | | |
| BUTYRATE | | | |
| ETHYL 2- | | 7452-79-1 | 0.1 |
| METHYL- | | | |
| BUTYRATE | | | |
| CLONAL | 1-cyanoundecane | 2437-25-4 | 0.1 |
| ETH | 3-ethoxy-4-hydroxy- | 121-32-4 | 0.1 |
| VANILLIN | benzaldehyde | | |

The fragrance contains 2.3% of a combination of phenylglycidate and 1,2-diketone in a ratio of phenylglycidate: 1,2-diketone of 6.67:1, and the malodor reducing composition of the invention comprises 0.23% of the final product composition.

### EXAMPLE 4

### A Gel Air Freshener

Table 11 gives the formulation of a water based gel air freshener to which the fragrance of the table 8 can be added for a malodor countering effect.

TABLE 11

| Ingredient | % |
|---|---|
| Deionised water | To 100 |
| Carageenan gum | 3.0 |
| Guar Gum | 0.3 |
| Preservative | 0.3 |
| Fragrance of table 8 | 4.0 |

Add the carageenan gum and Guar gum to water with continuous mixing. Heat the water to 80° C. with steady mixing until the polymers have dissolved. Allow to cool and add the fragrance and preservative at 60° C. with mixing. After 10 minutes pour into container. The phenylglycidate and 1,2-diketone malodor reducing composition comprise 3% of the fragrance of table 8 and thereby comprise 0.12% of the final air freshener gel.

### EXAMPLE 5

### Water Containing Aerosol Air Freshener

Table 12 gives the formulation of an aqueous air freshener spray into which fragrances of the invention such as the fragrance of table 8 can advantageously employed to reduce malodor.

TABLE 12
An Aqueous Air Freshener Composition

| Ingredient | % |
|---|---|
| Fragrance of table 8 corrected to 100% | 0.40 |
| Propellant (Propane/Butane) | 30.00 |
| Monosodium dihydrogen phosphate/Sodium | q.s. Approx 1% |
| Hydroxide pH buffer | |
| Sorbitan mono-oleate | 1.00 |
| Water | To 100% |

### EXAMPLE 6

### Liquid Rim Block

Table 13 gives an example of a toilet liquid rim block in which the fragrance of table 8 can be advantageously used to reduce malodor.

TABLE 13
Liquid Rim Block Composition

| Ingredient | Wt % |
|---|---|
| Emulgin HF70 (supplied by Cognis) | 16.7 |
| 1,2-Propylene glycol | 4.0 |
| Fragrance of table 8 | 6.0 |
| Kathon CG (supplied by Seppic) | 0.005 |
| Natrosol 250MR (supplied by Hercules) | 11.7 |
| Dyes and other minors | q.s. |
| Water | To 100 |

1. A malodor reducing composition comprising:
(A) phenylglycidate represented by the following formula (1): 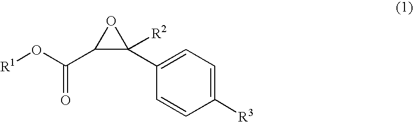
wherein R¹ is a C₁-C₄ branched or linear alkyl group, R² is a hydrogen atom or methyl group, and R³ is a hydrogen atom, a C₁-C₄ branched or linear alkyl group or a methoxyl group;
and (B) 1,2-diketone represented by the following formula (2) or (3): 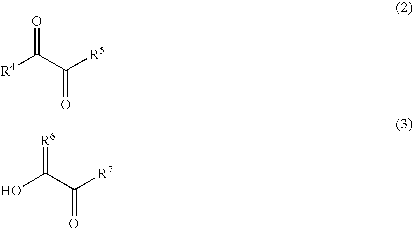
wherein R⁴, R⁵ and R⁷ are each independently a C₁-C₅ linear or branched alkyl or alkenyl group, R⁶ is a (C₁-C₅) alkylidene group, R⁴ and R⁵ may form a C₄-C₇ saturated or unsaturated alicyclic or hetrocyclic ring structure which may be mono-substituted or poly-substituted by (C₁-C₄) alkyl groups, and R⁶ and R⁷ may form a C₄-C₇ unsaturated, alicyclic or heterocyclic ring structure which may be mono-substituted or poly-substituted by (C₁-C₄) alkyl groups;
a weight ratio of the phenylglycidate (A) to the 1,2-diketone (B) being from 1:99 to 99:1.

2. The malodor reducing composition according to claim 1, wherein the phenylglycidate is selected from the group consisting of ethyl 3-phenylglycidate (121-39-1), ethyl 3-methyl-3-phenylglycidate (77-83-8) and mixtures thereof.

3. The malodor reducing composition according to claim 1, wherein the 1,2-diketone is selected from the group consisting of β-thujaplicine (499-44-5), 3-methylcyclopent-2-en-2-ol-1-one (765-70-8), 2,3-butanedione (431-03-8), 2-ethyl-3-hydroxypyran-4-one (4940-11-8), 2-methyl-3-hydroxypyran-4-one (118-71-8), ethyl cyclopentenolone (21835-01-8), 3,4-dimethyl-1,2-cyclopentadione (13494-06-9), 3,5-dimethyl-1,2-cyclopentadione (13494-07-0), 3-ethyl-2-hydroxy-4-methylcyclopent-2-en-1-one (42348-12-9), 2-hydroxycyclohex-2-en-1-one (10316-66-2), 1-methyl-2,3-cyclohexadione (3008-43-3), 1,2-cyclohexadione (765-87-7) and mixtures thereof.

4. The malodor reducing composition according to claim 3, wherein the 1,2-diketone is selected from the group consisting of β-thujaplicine (499-44-5), 2,3-butanedione (431-03-8), 2-ethyl-3-hydroxypyran-4-one (4940-11-8), 2-methyl-3-hydroxypyran-4-one (118-71-8) and mixtures thereof.

5. A fragrance composition comprising at least 0.05% by weight of the malodor reducing composition according to claim 1 based on a total composition of the fragrance composition.

6. The fragrance composition according to claim 5, which further contains more than 10% by weight of at least one of aldehydes and α,β-unsaturated aldehydes based on a total composition of the fragrance composition.

7. A product comprising the malodor reducing composition according to claim 1 in an amount of 0.00005% to 15% by weight based on a total composition of said product.

8. The product according to claim 7, which is selected from the group consisting of air care products, personal cleansing and deodorizing products, household cleaning products including cistern blocks, rim blocks or liquid rim blocks, and laundry and fabric care products.

9. A product comprising the fragrance composition according to claim 5 in an amount of 0.05% to 100% by weight based on a total composition of the product.

10. The product according to claim 9, which is selected from the group consisting of personal cleansing products, household cleaning products including cistern blocks, rim blocks or liquid rim blocks, and laundry and fabric care products, and comprises 0.05% to 15% by weight of the fragrance composition based on a total composition of the product.

11. The product according to claim 9, which is an air care product.

12. The product according to claim 9, which comprises the fragrance composition in an amount of 0.05% to 1.5% by weight based on a total composition of the product.

13. The product according to claim 9, which is selected from the group consisting of personal cleansing and deodorizing products, and household cleaning products including cistern and rim blocks and liquid rim blocks.
